(19) United States
(12) Patent Application Publication
(10) Pub. No.: US 2017/0252101 A1
(43) Pub. Date: Sep. 7, 2017

(54) SYSTEMS AND METHODS FOR INTRAPROCEDURAL EVALUATION OF RENAL DENERVATION

(71) Applicant: St. Jude Medical, Cardiology Division, Inc., St. Paul, MN (US)

(72) Inventors: Cary Hata, Irvine, CA (US); William Vu, Laguna Niguel, CA (US); Yongxing Zhang, Irvine, CA (US); Tho Nguyen, Huntington Beach, CA (US); Vivian Tran, San Juan Capistrano, CA (US); Irvin Narciso, Tustin, CA (US); Rita DeRama, Irvine, CA (US)

(21) Appl. No.: 15/061,243

(22) Filed: Mar. 4, 2016

(51) Int. Cl. A61B 18/14; A61B 5/026; A61B 5/024; A61N 1/36; A61B 5/021

(52) U.S. Cl. CPC A61B 18/1492; A61N 1/36007; A61B 5/021; A61B 5/024; A61B 5/02405; A61B 5/026; A61B 2018/1467; A61B 2018/00577

(57) ABSTRACT

The present disclosure provides methods, systems, and apparatuses for providing real time intraprocedural operational feedback to the operator as to the success of a renal denervation procedure and its overall effectiveness for reducing the blood pressure of a subject to allow for more precise and thorough ablation of the renal artery and better patient outcomes. Using this real time feedback generated through the electrical stimulation of multiple points about the circumference of the renal artery at specific times during the procedure, the operator can assess the desired procedural endpoint and whether the amount of renal denervation provided to the subject is sufficient, or whether the subject may benefit from further denervation of the renal artery. The present disclosure provides means for providing real time intraprocedural feedback to the operator that can be incorporated into conventional renal denervation catheters and equipment.

### A. FIELD OF THE DISCLOSURE

The present disclosure relates generally to systems and methods for the intraprocedural evaluation of the endpoint of a renal denervation procedure. In particular, the present disclosure relates to systems and methods for determining the intraprocedural confirmation of an effective renal denervation procedure by evaluating a particular physiological response of a subject prior to and after a renal denervation procedure wherein the physiological response is generated through the electrical stimulation of multiple points about the circumference of a portion of the subject's renal artery. Other methods of the present disclosure relate to patient screening and evaluation for renal denervation procedures.

### B. BACKGROUND

Hypertension remains the most prevalent cardiovascular risk factor around the globe today. In humans, sympathetic nerve activity is increased in almost all forms of hypertension. Various renal denervation ablation procedures for the ablation of perivascular renal nerves in the renal arteries have been used for the treatment of hypertension, and specifically for drug-resistant hypertension. Generally, one or more radiofrequency electrodes are introduced into the body and fed into the renal artery and used to ablate the efferent and afferent nerves that generally run the length of the artery. In some cases, a single ablation procedure may include six to ten or more ablation areas along and around the wall of the artery. Typically, the operator performing the procedure will ablate one discrete area of the artery and then move the ablation electrode a desired distance lengthwise about the length of the artery and also rotate the handle of the catheter to move the ablation electrode circumferentially around the artery. In some cases, the operator may move the ablation electrode circumferentially about 45 degrees around the artery wall between ablations. By varying the ablation treatment sites lengthwise down and circumferentially around the artery wall, any potential overall damage to the artery wall can be minimized or eliminated while the overall ablation of the efferent and afferent nerves can still be substantially complete and effective.

During the ablation procedure, the operator, typically a doctor, performing the procedure generally attempts to monitor and track all of the areas of the artery wall that have previously been ablated to avoid over-treatment of any one site. This monitoring and tracking is generally done both along the length of the artery as well as around the circumference of the artery wall to ensure proper ablation of the arterial nerves and the best procedural results. Feedback to the operator is generally provided regarding the temperature at the ablation site, which can also be indicative of the effectiveness of the ablation procedure itself, and whether the nerve has been ablated to a desired endpoint.

### BRIEF SUMMARY OF THE DISCLOSURE

The present disclosure provides methods, systems, and apparatuses for providing real time intraprocedural operational feedback to the operator as to the endpoint of a renal denervation procedure to allow for more precise and thorough ablation of the renal artery and better patient outcomes. Using this real time feedback generated through the electrical stimulation of multiple points about the circumference of at least a portion of the renal artery at specific times during the procedure, the operator can assess the desired procedural endpoint and whether the amount of renal denervation provided to the subject is sufficient, or whether the subject may benefit from further denervation of the renal artery. In some embodiments, the electrical stimulation described herein will occur at one point in each of the four quadrants of at least a portion of the renal artery. The present disclosure provides means for providing real time intraprocedural feedback via a subject's physiological responses to electrical stimulation to the operator that can be incorporated into conventional renal denervation catheters and equipment. Various embodiments of the present disclosure are set forth herein.

In one embodiment, the present disclosure is directed to a method of evaluating the intraprocedural success of a renal denervation procedure on a subject. The method comprises: (i) electrically stimulating multiple points about the circumference of a renal artery of the subject to determine a baseline physiological response of the subject; (ii) performing a renal denervation procedure on the renal artery of the subject; (iii) electrically stimulating the multiple points about the circumference of the renal artery of the subject to determine the physiological response of the subject; and (iv) comparing the baseline physiological response of the subject prior to renal denervation with the physiological response after renal denervation.

In another embodiment, the present disclosure is directed to a method of determining the end point of a renal denervation procedure on a subject. The method comprises: (i) electrically stimulating multiple points about the circumference of a renal artery of the subject through electrical stimulation to determine a baseline reading of the subject's blood pressure using an ablation catheter comprising at least four pairs of ablation electrodes and electrical stimulation electrodes and configured to electrically contact the renal artery, wherein the distance between each ablation electrode and each electrical stimulation electrode within the pairs is from about 0.5 millimeters to about 2.5 millimeters; (ii) performing a renal denervation procedure on the renal artery of the subject using the ablation catheter; (iii) electrically stimulating the multiple points about the circumference of the renal artery of the subject through electrical stimulation using an ablation catheter configured to electrically contact the renal artery to determine the subject's blood pressure; (iv) comparing the baseline blood pressure of the subject prior to renal denervation with the blood pressure after renal denervation; and (v) determining whether to perform an additional renal denervation procedure.

In another embodiment, the present disclosure is directed to a method of screening a subject for a renal denervation procedure. The method comprises electrically stimulating the four quadrants of a renal artery of the subject through electrical stimulation using an ablation catheter configured to electrically contact the four quadrants of the renal artery to determine a baseline reading of the subject's blood pressure and determining whether the baseline reading meets a threshold indicating that the subject may benefit from a renal denervation procedure.

In another embodiment, the present disclosure is directed to a method of screening a subject for a renal denervation procedure. The method comprises: (i) electrically stimulating the four quadrants of a renal artery of the subject through sequential or simultaneous electrical stimulation using an ablation catheter configured to electrically contact the four quadrants of the renal artery to determine a baseline reading of the subject's blood pressure; (ii) performing a renal denervation procedure on the renal artery of the subject using the ablation catheter; (iii) electrically stimulating the four quadrants of the renal artery of the subject through sequential or simultaneous electrical stimulation using an ablation catheter configured to electrically contact the four quadrants of the renal artery to determine the subject's blood pressure; (iv) comparing the baseline blood pressure of the subject prior to renal denervation with the blood pressure after renal denervation; and (v) determining whether the subject may benefit from additional renal denervation.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagrammatic view of a renal denervation system for presenting information relating to lesion formation in a renal artery.

FIG. 2 is a diagram of an electrode basket including an ablation electrode and an electrical stimulation electrode on each spline suitable for use in the present processes.

FIG. 3 is a diagram of an electrode basket including a single ablation electrode on each spline suitable for use in the present processes.

FIG. 4 is a diagram of an electrode basket including two ablation electrodes on each spline suitable for use in the present processes.

FIG. 5 is a flow chart of one embodiment of a method for evaluating the intraprocedural success of a renal denervation procedure on a subject.

FIG. 6 is a flow chart of one embodiment of a method of determining the end point of a renal denervation procedure on a subject.

FIG. 7 is flow chart of one embodiment of a method of screening a subject for a renal denervation procedure.

FIG. 8 is a flow chart of another embodiment of a method of screening a subject for a renal denervation procedure.

FIG. 9 is a block diagram of a radiofrequency ablation generator modified to include electrical stimulation.

FIG. 10 is a graph illustrating the change in blood pressure (for systolic, diastolic, and mean) for numerous electrical stimulations of a canine in Example 1.

FIGS. 11-13 are graphs illustrating the change in blood pressure (for systolic, diastolic, and mean) before and after one or two renal denervation procedures for a canine in Example 2.

Corresponding reference characters indicate corresponding parts throughout the several views of the drawings. It is understood that that Figures are not necessarily to scale.

### DETAILED DESCRIPTION OF THE DISCLOSURE

The present disclosure provides methods, systems, and apparatuses for providing real time feedback to an operator, such as a doctor, performing a renal denervation procedure on a subject. In many embodiments, methods are provided for evaluating the intraprocedural confirmation of the end point of a renal denervation procedure such that the operator can assess the effectiveness of a procedure and decide whether additional renal denervation may be beneficial to the subject. Methods are also provided for screening subjects to determine whether the subject might benefit from a renal denervation procedure. Many methods of the present disclosure utilize a modified ablation catheter including electrical stimulation electrodes in combination with ablation electrodes for electrical stimulation of multiple points about the circumference of the renal artery to provide intraprocedural feedback to the operator. In many embodiments, the multiple points about the circumference of the renal artery will include at least one point in each of the four quadrants of the renal artery. As used herein, “quadrant” refers to a circular section equal to one quarter of a circle such that the renal artery, which is generally circular in cross-section, would include four quadrants. Other embodiments of the present disclosure utilize an ablation catheter including one or more ablation electrodes per catheter basket-spline that are capable of providing ablation and electrical stimulation in the absence of electrical stimulation electrodes.

More specifically, the embodiments of the present disclosure utilize simultaneous or sequential electrical stimulation of multiple points about the circumference of the renal artery, or section thereof, prior to a renal denervation procedure to establish a baseline physiological response (such as a baseline blood pressure, baseline heart rate, baseline renal blood flow, or other physiological response as described herein) of a subject prior to any renal denervation taking place in the renal artery. By using electrical stimulation of multiple points about the circumference of the renal artery, the physiological response is amplified due to the cumulative response of the multiple-point electrical stimulation as further described herein. The present methods significantly increase the likelihood of obtaining the true and accurate physiological response due to the electrical stimulation of multiple points about the circumference of the renal artery. Once the baseline physiological response has been established after electrical stimulation, the operator can perform a renal denervation procedure on the renal artery and then subsequently electrically stimulate the multiple points about the circumference of the renal artery again to measure the physiological response of the subject after the renal denervation is complete. With this information, the operator can compare the physiological response before the renal denervation (after the first electrical stimulation) with the blood pressure after renal denervation (after the second electrical stimulation), and determine, intraprocedurally, the effectiveness of the procedure and whether the subject may benefit from further denervation. The operator can also minimize both the number of ablations performed and the energy used, which can reduce or eliminate any potential damage to the renal artery and improve patient outcomes. Additionally, the real time information may allow for improved overall procedure management and efficiency. The methods of the present disclosure may further be utilized to screen potential renal denervation subjects to segregate those that might benefit from a renal denervation procedure from those that likely would not benefit.

The efferent and afferent nerves that lie within and immediately adjacent to the wall of the renal artery propagate longitudinally down the length of the renal wall in a highly non-uniform, irregular pattern that may be sometimes described as a spider-web or highly variable and non-predictable pattern. As such, it is generally difficult to determine the exact position of the efferent and afferent nerves of the renal artery. The methods of the present disclosure account for this highly irregular and unpredictable longitudinal propagation of the efferent and afferent nerves through the electrical stimulation of multiple points about the circumference of the renal artery as described herein to elucidate the desired physiological response at the desired time of the procedure. By electrically stimulating at multiple points about the circumference of the renal artery, the ability to achieve sufficient electrical stimulation of the efferent and afferent nerves, and thus elicit a significant and measurable impact on the desired physiological response being evaluated, is significantly increased as described herein and further shown in Example 1. The multiple point electrical stimulation results in a substantially increased likelihood of the electrical stimulation interacting with the desired nerves as a greater area of the renal artery is electrically stimulated. In many embodiments, it is desirable to have electrical stimulation occur in at least one location in each of the four quadrants of the renal artery; that is, it is desirable in some embodiments to electrically stimulate at least one location in each of the four quadrants of the renal artery. In many embodiments, the electrical stimulation points about the circumference of the renal artery may be equally spaced apart to reduce the potential of any damage to the renal artery and/or the efferent and afferent nerves.

For purposes of this description, the methods, systems, and apparatuses of the present disclosure will be primarily described in connection with the use of an ablation catheter having four splines, with each spline including thereon one ablation electrode and one electrical stimulation electrode, or one or two ablation electrodes and zero electrical stimulation electrodes. It is contemplated, however, that numerous other types of ablation catheters, including catheters having 5, 6, 7, 8, 9, 10 or more splines can be used in the methods of the present disclosure as would be appreciated by one of ordinary skill in the art based on the disclosure herein. Additionally, it is also contemplated that the electrical stimulation within the renal artery described herein could be provided by an independent system not integral with the ablation catheter.

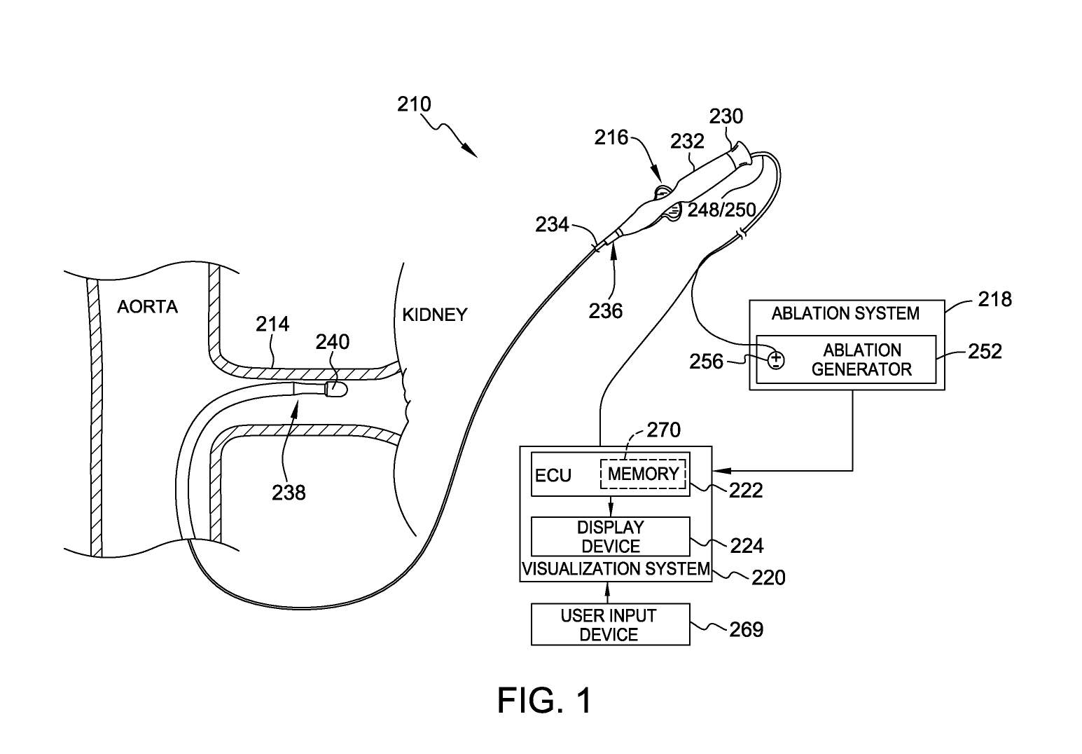

Referring now to the Figures, FIG. 1 illustrates one exemplary embodiment of a general ablation system 210 for performing one or more diagnostic and/or therapeutic functions that include components for presenting information representative of lesion formations in renal artery 214 during an ablation procedure performed thereon.

Among other components, system 210 includes a medical device (such as, for example, catheter 216), ablation system 218, and system 220 for the visualization, navigation, and/or mapping of internal body structures. System 220 may include, for example and without limitation, an electronic control unit (ECU) 222, display device 224, user input device 269, and memory 270. Alternatively, ECU 222 and/or display device 224 may be separate and distinct from, but electrically connected to and configured for communication with, system 220.

With continued reference to FIG. 1, catheter 216 is provided for examination, diagnosis, and/or treatment of internal body tissues, such as renal artery 214. In an exemplary embodiment, catheter 216 comprises a radio frequency (RF) ablation catheter. It should be understood, however, that catheter 216 is not limited to an RF ablation catheter. Rather, in other embodiments, catheter 216 may comprise an irrigated catheter and/or other types of ablation catheters (e.g., cryoablation, ultrasound, etc.).

In an exemplary embodiment, catheter 216 is electrically connected to ablation system 218 to allow for the delivery of RF energy. Catheter 216 may include a cable connector or interface 230, handle 232, shaft 234 having a proximal end 236 and distal end 238 (as used herein, “proximal” refers to a direction toward the end of catheter 216 near the operator, and “distal” refers to a direction away from the operator and (generally) inside the body of a subject or patient), and one or more electrodes 240 mounted in or on shaft 234 of catheter 216. In an exemplary embodiment, electrode 240 is disposed at or near distal end 238 of shaft 234, with electrode 240 comprising an ablation electrode disposed at the extreme distal end 238 of shaft 234 for contact with renal artery 214. Catheter 216 may further include other conventional components such as, for example and without limitation, sensors, additional electrodes (e.g., ring electrodes) and corresponding conductors or leads, or additional ablation elements, e.g., a high intensity focused ultrasound ablation element and the like.

Connector 230 provides mechanical and electrical connection(s) for cables 248 and 250 extending from ablation system 218, and visualization, navigation, and/or mapping system 220. Connector 230 is conventional in the art and is disposed at the proximal end of catheter 216.

Handle 232 provides a location for the operator to hold catheter 216 and may further provide means for steering or guiding shaft 234 within renal artery 214. For example, handle 232 may include means to change the length of a guidewire extending through catheter 216 to distal end 238 of shaft 234 to steer shaft 234. Handle 232 is also conventional in the art and it will be understood that the construction of handle 232 may vary. In another exemplary embodiment, catheter 216 may be robotically driven or controlled. Accordingly, rather than an operator manipulating a handle to steer or guide catheter 216, and shaft 234 thereof, in particular, a robot is used to manipulate catheter 216.

Shaft 234 is generally an elongated, tubular, flexible member configured for movement within renal artery 214. Shaft 234 supports, for example and without limitation, electrode 240, associated conductors, and possibly additional electronics used for signal processing or conditioning. Shaft 234 may also permit transport, delivery and/or removal of fluids (including irrigation fluids, cryogenic ablation fluids, and bodily fluids), medicines, and/or surgical tools or instruments. Shaft 234 may be made from conventional materials such as polyurethane, and defines one or more lumens configured to house and/or transport at least electrical conductors, fluids, or surgical tools. Shaft 234 may be introduced into renal artery 214 through a conventional introducer. Shaft 234 may then be steered or guided through renal artery 214 to a desired location with guidewires or other means known in the art.

With further reference to FIG. 1, ablation system 218 is comprised of, for example, ablation generator 252. Ablation generator 252 generates, delivers, and controls RF energy output by ablation catheter 216 and electrode 240 thereof, in particular. In an exemplary embodiment, generator 252 includes RF ablation signal source 256 configured to generate an ablation signal that is output across a pair of source connectors: a positive polarity connector SOURCE (+), which may be electrically connected to tip electrode 240 of catheter 216; and a negative polarity connector SOURCE (−). It should be understood that the term connectors as used herein does not imply a particular type of physical interface mechanism, but is rather broadly contemplated to represent one or more electrical nodes. Source 256 is configured to generate a signal at a predetermined frequency in accordance with one or more user specified parameters (e.g., power, time, etc.) and under the control of various feedback sensing and control circuitry as is known in the art. Source 256 may generate a signal, for example, with a frequency of about 450 kHz or greater. Generator 252 may also monitor various parameters associated with the ablation procedure including, for example, impedance, the temperature at the distal tip of the catheter, applied ablation energy, and the position of the catheter, and provide feedback to the clinician or another component within system 210 regarding these parameters.

In accordance with one embodiment of the present disclosure, some ablation catheters suitable for use in the processes and systems described herein for the evaluation of the intraprocedural success of a renal denervation procedure and/or patient screening include both ablation electrodes and electrical stimulation electrodes thereon capable of contacting the interior of a renal artery after insertion therein. In other embodiments of the present disclosure, some ablation catheters suitable for use in the processes and systems described herein for the evaluation of the intraprocedural success of a renal denervation procedure and/or patient screening include only ablation electrodes that are capable of providing an ablation function as well as an electrical stimulation function. The exact type and design of the ablation catheter is not critical so long as the ablation catheter is configured to allow for electrical stimulation at multiple points about the circumference of the renal artery. In some embodiments, the ablation catheter is sized and configured to allow for electrical stimulation of at least 2, or even 3, or even 4 or even 5 or even 6 or even 7 or even 8 or more points about the circumference of the renal artery. In many embodiments, the ablation catheter is sized and configured such that the electrical stimulation at multiple points is generally equally spaced apart about the circumference of the renal artery. In one specific embodiment, the ablation catheter is sized and configured to allow for electrical stimulation at one point within each of the four quadrants of the renal artery. Such four quadrant electrical stimulation may desirably be equally spaced apart in many embodiments. Many types of ablation catheters may be configured in this manner and are suitable for use in accordance with the present disclosure including, for example, multiple spline catheters (i.e., electrode baskets having 2, 3, 4, 5, 6, or more splines), spiral catheters, balloon catheters, and the like).

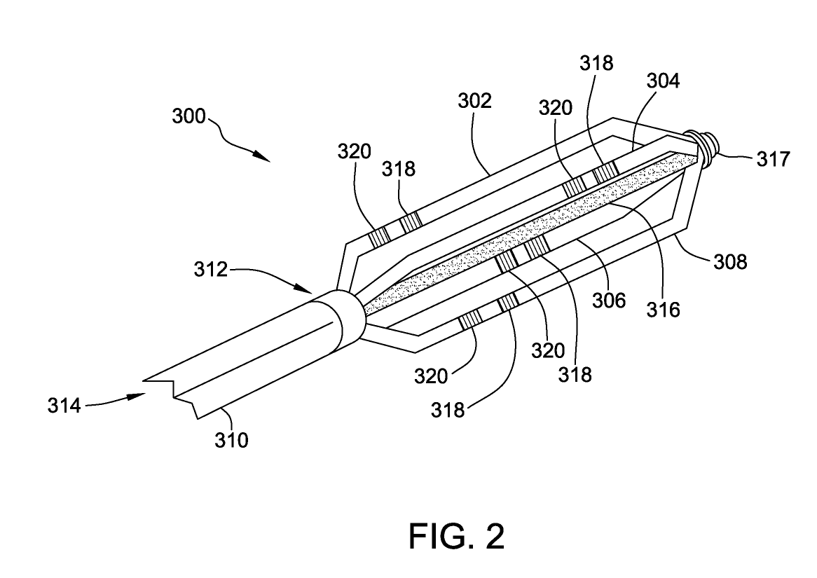

Referring now to FIG. 2, there is illustrated an electrode basket in accordance with one embodiment of the present disclosure that is suitable for use with an ablation catheter in the methods of the present disclosure. Electrode basket 300 includes four splines 302, 304, 306, and 308 connected to an elongated catheter body 310 having distal end 312 and proximal end 314. A pulling wire 316 is located in the center of electrode basket 300 and is attached to junction 317. Pulling wire 316 can be used to expand/contract electrode basket 300 into different conformations before, during, and after an ablation procedure. Splines 302, 304, 306, and 308 of electrode basket 300 each include ablation electrodes 318 and electrical stimulation electrodes 320. Ablation electrodes 318 and electrical stimulation electrodes 320 are positioned on splines 302, 304, 306, and 308 to allow contact thereof with each quadrant of a renal artery (not shown in FIG. 2 but see FIG. 1) after insertion and expansion therein.

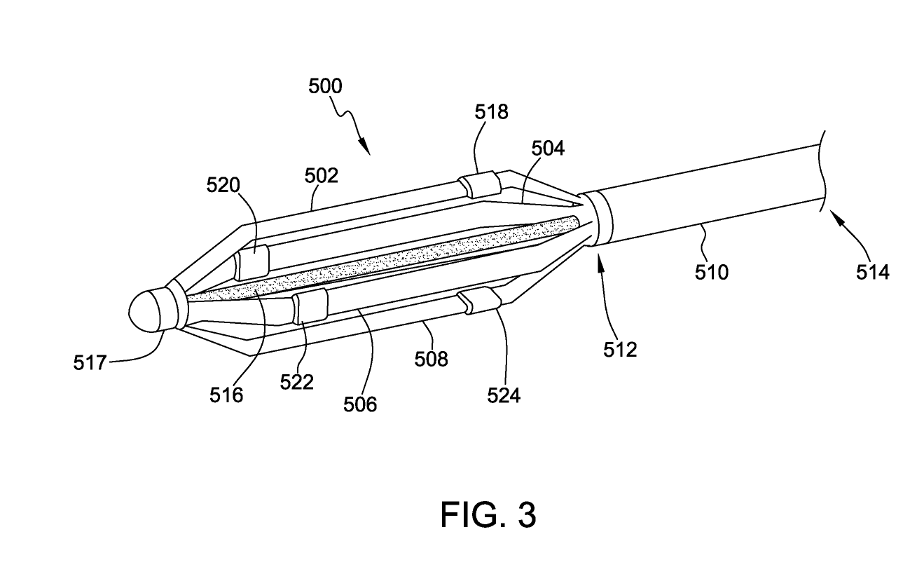

Referring now to FIG. 3, there is illustrated an electrode basket in accordance with one embodiment of the present disclosure that is suitable for use with an ablation catheter in methods of the present disclosure. Electrode basket 500 includes four splines 502, 504, 506, and 508 connected to an elongated catheter body 510 having distal end 512 and proximal end 514. A pulling wire 516 is located in the center of electrode basket 500 and is attached to junction 517. Pulling wire 516 can be used to expand/contract electrode basket 500 into different conformations before, during, and after an ablation procedure. Splines 502, 504, 506, and 508 of electrode basket 500 each include a single ablation electrode 518, 520, 522, and 524, respectfully, that is capable of providing an ablation function and an electrical stimulation function as described herein. Ablation electrodes 518, 520, 522, and 524 are positioned on splines 502, 504, 506, and 508 to allow contact thereof with each quadrant of a renal artery (not shown in FIG. 3 but see FIG. 1) after insertion and expansion therein. In this embodiment illustrated in FIG. 3, paired electrical stimulation (using 2 out of the 4 pairs of electrodes) (bipolar electrical stimulation) may be utilized to provide the electrical stimulation described herein. In many embodiments, the pairs are sequentially stimulated, although simultaneous stimulation is within the scope of the present disclosure. In one exemplary embodiment, the bipolar electrical stimulation is provided through sequential electrical stimulation of electrodes 520 and 522, followed by electrodes 520 and 518, followed by electrodes 522 and 524, and followed by electrodes 518 and 524. Other bipolar sequential stimulation patterns are also within the scope of the present disclosure.

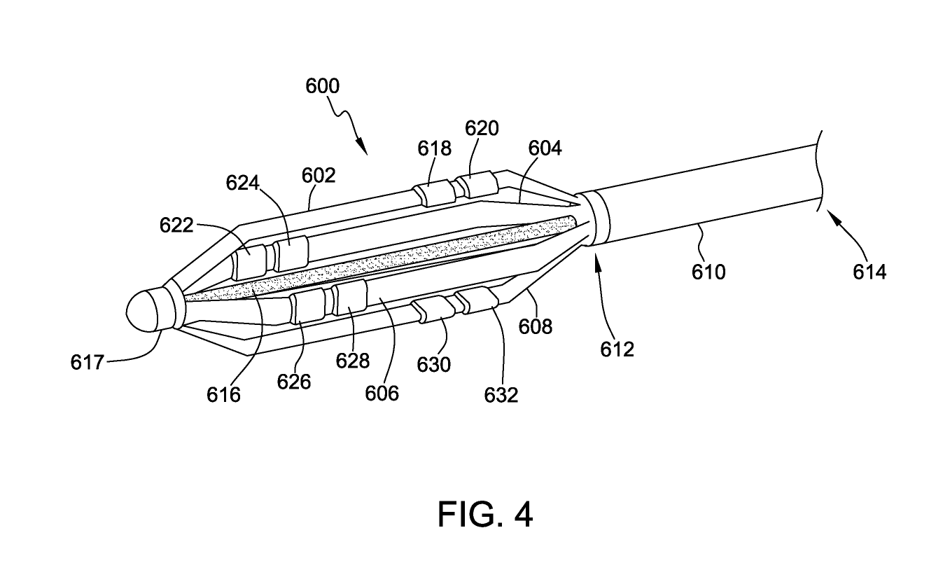

Referring now to FIG. 4, there is illustrated an electrode basket in accordance with one embodiment of the present disclosure that is suitable for use with an ablation catheter in methods of the present disclosure. Electrode basket 600 includes four splines 602, 604, 606, and 608 connected to an elongated catheter body 610 having distal end 612 and proximal end 614. A pulling wire 616 is located in the center of electrode basket 600 and is attached to junction 617. Pulling wire 616 can be used to expand/contract electrode basket 600 into different conformations before, during, and after an ablation procedure. Splines 602, 604, 606, and 608 of electrode basket 600 each include two ablation electrodes: 618 and 620 on spline 602; 622 and 624 on spline 604; 626 and 628 on spline 606; and 630 and 632 on spline 608. These ablation electrodes are capable of providing an ablation function and an electrical stimulation function as described herein. The ablation electrodes are positioned on splines 602, 604, 606, and 608 to allow contact thereof with each quadrant of a renal artery (not shown in FIG. 4 but see FIG. 1) after insertion and expansion therein. In this embodiment illustrated in FIG. 4, paired electrical stimulation (using 2 out of the 4 pairs of electrodes, with the electrode pair being located on the same spline) (bipolar electrical stimulation) may be utilized to provide the electrical stimulation described herein. In many embodiments, the pairs are sequentially stimulated, although simultaneous stimulation is within the scope of the present disclosure. In one exemplary embodiment, the bipolar electrical stimulation is provided through sequential electrical stimulation of electrodes 622 and 624, followed by electrodes 626 and 628, followed by electrodes 618 and 620, and followed by electrodes 630 and 632. Other bipolar sequential stimulation patterns are also within the scope of the present disclosure.

As discussed in further detail herein, the electrical stimulation provided by the electrical stimulation electrodes and/or the ablation electrodes described herein is desirably at an intensity level sufficient to elicit the desired physiological response in the subject, but is not too intense as to potentially damage the renal artery or nerves therein or introduce a high level of pain to the subject; that is, the electrical stimulation provided to the multiple points about the circumference of the renal artery by the electrical stimulation and/or ablation electrodes should not be too diffuse or too intense. In many embodiments where both ablation electrodes and electrical stimulation electrodes are present on the same spline (or two ablation electrodes are present on the same spline), in order to ensure that the desired amount of electrical stimulation is provided by the electrodes, in many embodiments, including the multiple spline electrode basket embodiment described above, it is desirable to position the ablation electrode and the electrical stimulation electrode (or the ablation electrodes) such that the distance between them is from about 0.5 millimeters to about 2.5 millimeters, desirably from about 1.0 millimeter to about 2.0 millimeters.

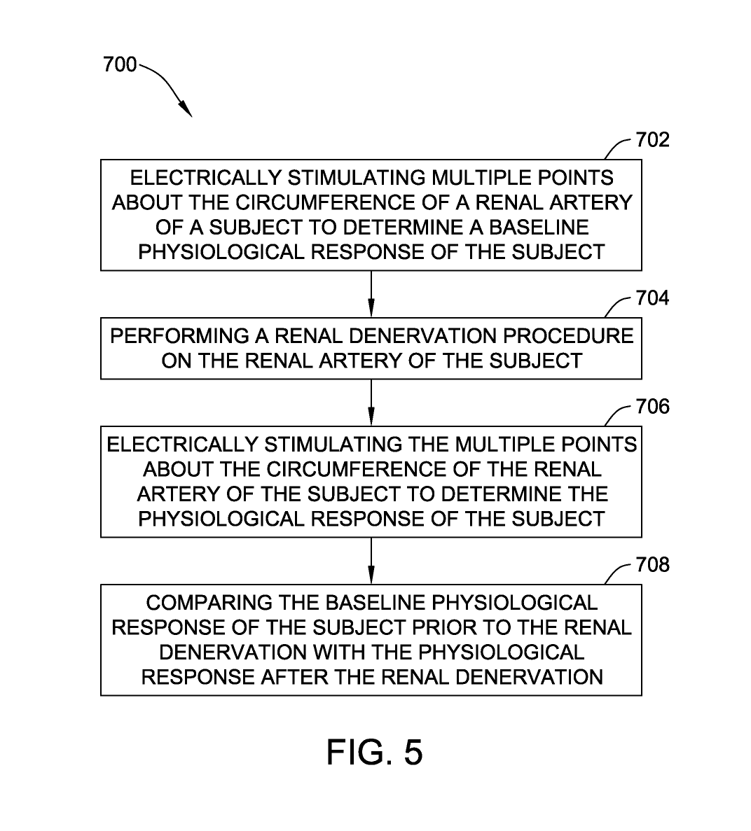

In accordance with various embodiments of the present disclosure, ablation catheters including ablation electrodes capable of providing ablation and electrical stimulation functionality and ablation catheters including both ablation electrodes and electrical stimulation electrodes may be used in methods described herein for the evaluation of the intraprocedural success of a renal denervation procedure on a subject; that is, electrical stimulation to the renal artery at prescribed times as described herein may be used to provide real time feedback to the operator of a renal denervation procedure on a subject to allow the operator to determine how successful a renal denervation procedure on the subject has been, and determine whether further denervation may be beneficial or desirable. Other embodiments allow for methods of screening patients for renal denervation procedures. A number of exemplary methods of the present disclosure are set forth in FIGS. 5-8 and described in more detail hereinbelow.

FIG. 5 is a flow chart of one embodiment of a method 700 for evaluating the intraprocedural success of a renal denervation procedure. Method 700 includes electrically stimulating 702 multiple points about the circumference of a renal artery of a subject to determine a baseline physiological response of the subject; performing 704 a renal denervation procedure on the renal artery of the subject; electrically stimulating 706 the multiple points about the circumference of the renal artery of the subject to determine the physiological response of the subject; and comparing 708 the baseline physiological response of the subject prior to the renal denervation with the physiological response after the renal denervation.

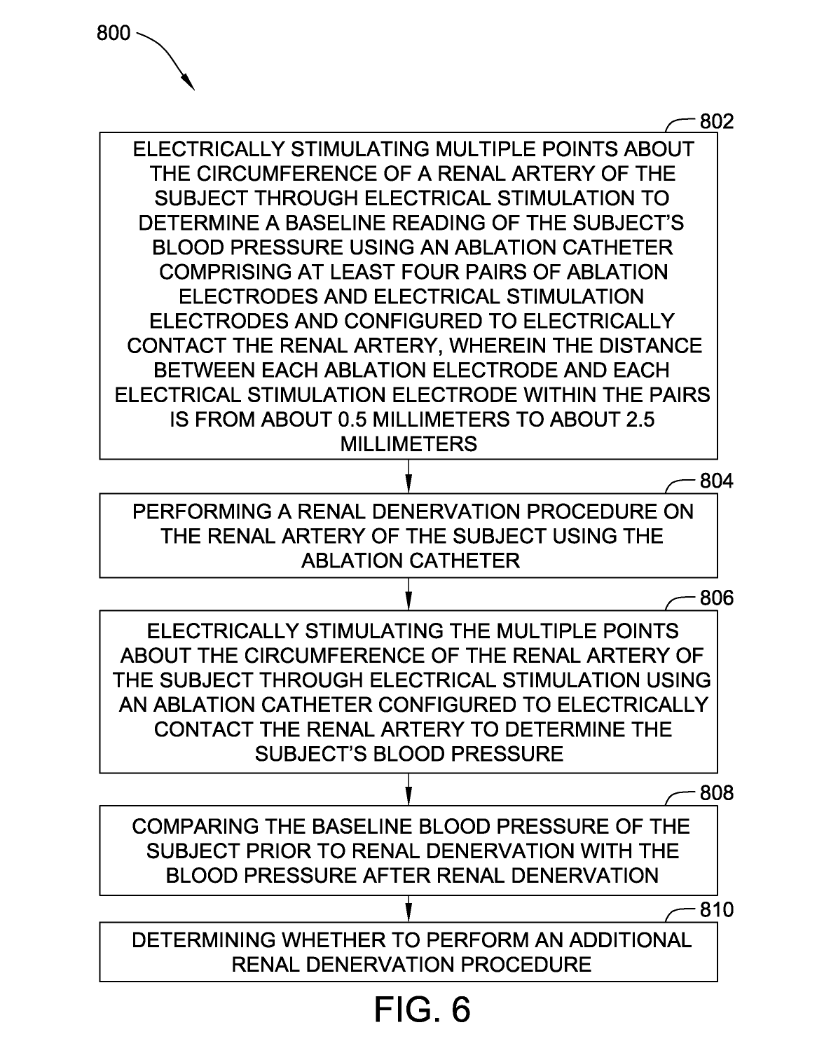

FIG. 6 is a flow chart of one embodiment of a method 800 for determining the end point of a renal denervation procedure on a subject. Method 800 includes electrically stimulating 802 multiple points about the circumference of a renal artery of the subject through electrical stimulation to determine a baseline reading of the subject's blood pressure using an ablation catheter comprising at least four pairs of ablation electrodes and electrical stimulation electrodes and configured to electrically contact the renal artery, wherein the distance between each ablation electrode and each electrical stimulation electrode within the pairs is from about 0.5 millimeters to about 2.5 millimeters; performing 804 a renal denervation procedure on the renal artery of the subject using the ablation catheter; electrically stimulating 806 the multiple points about the circumference of the renal artery of the subject through electrical stimulation using an ablation catheter configured to electrically contact the renal artery to determine the subject's blood pressure; comparing 808 the baseline blood pressure of the subject prior to renal denervation with the blood pressure after renal denervation; and determining 810 whether to perform an additional renal denervation procedure.

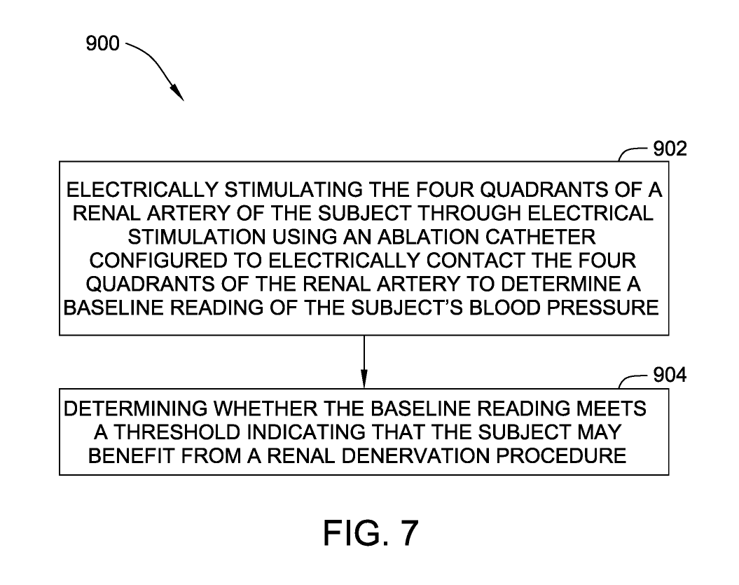

FIG. 7 is a flow chart of one embodiment of a method 900 of screening a subject for a renal denervation procedure. Method 900 includes electrically stimulating 902 the four quadrants of a renal artery of the subject through electrical stimulation using an ablation catheter configured to electrically contact the four quadrants of the renal artery to determine a baseline reading of the subject's blood pressure and determining 904 whether the baseline reading meets a threshold indicating that the subject may benefit from a renal denervation procedure.

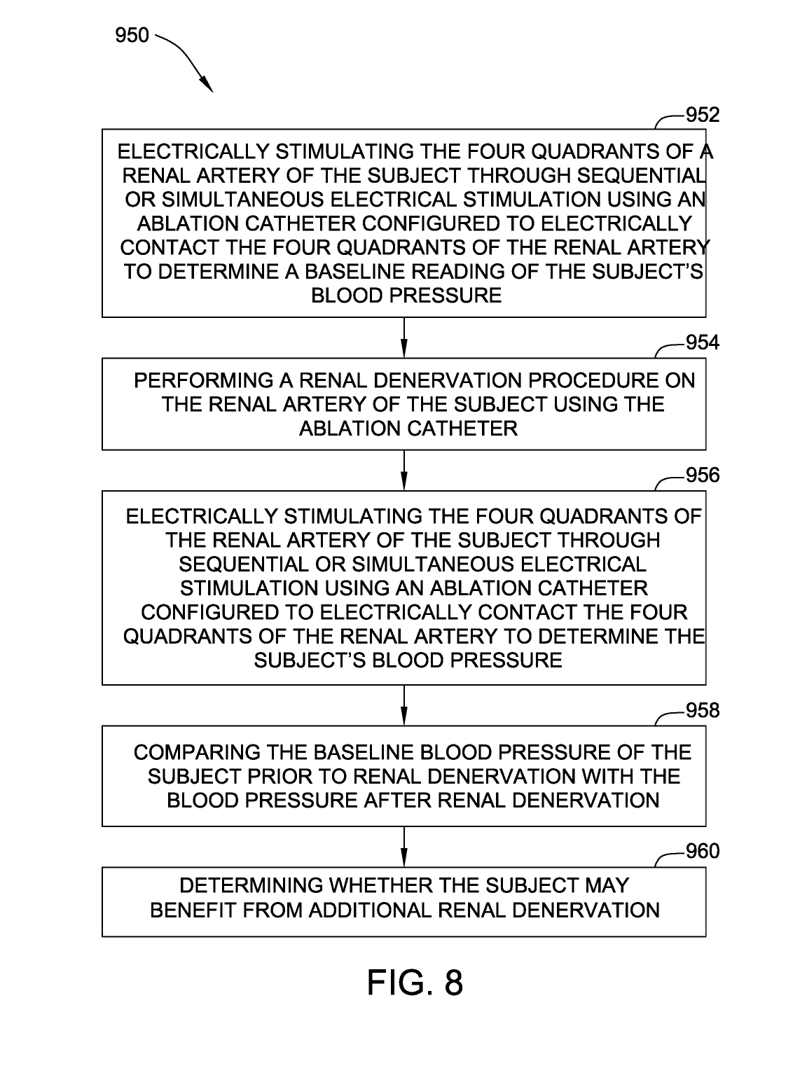

FIG. 8 is a flow chart of one embodiment of a method 950 of screening a subject for a renal denervation procedure. Method 950 includes electrically stimulating 952 the four quadrants of a renal artery of the subject through sequential or simultaneous electrical stimulation using an ablation catheter configured to electrically contact the four quadrants of the renal artery to determine a baseline reading of the subject's blood pressure; performing 954 a renal denervation procedure on the renal artery of the subject using the ablation catheter; electrically stimulating 956 the four quadrants of the renal artery of the subject through sequential or simultaneous electrical stimulation using an ablation catheter configured to electrically contact the four quadrants of the renal artery to determine the subject's blood pressure; comparing 958 the baseline blood pressure of the subject prior to renal denervation with the blood pressure after renal denervation; and determining 960 whether the subject may benefit from additional renal denervation.

In order to evaluate the intraprocedural success of a renal denervation procedure and/or perform patient screening, a baseline physiological response of the subject is first measured and recorded prior to any renal denervation occurring on the subject. A “baseline physiological response” refers to the response elicited by a subject after electrical stimulation of the multiple points about the circumference of the renal artery; for example, a baseline blood pressure would be the blood pressure determined after electrical stimulation. A number of physiological responses may be selected for measuring prior to the renal denervation procedure to establish a baseline reading that is later compared against another reading after renal denervation as described herein. Further examples include blood pressure, heart rate, renal blood flow, catecholamine (epinephrine and norepinephrine) level, heart rate variability, natriuresis, and other biomarker levels, and combinations thereof. In many embodiments, blood pressure is a desired physiological response to measure as a baseline as it can easily be measured outside of the subject's body throughout the course of the procedure. As used herein, the term “blood pressure” encompasses systolic blood pressure, diastolic blood pressure and mean blood pressure. Any of systolic blood pressure, diastolic blood pressure and mean blood pressure may be suitably used as a physiological response in accordance with the present disclosure. In some embodiments, two or more physiological responses may be measured.

The baseline physiological response of the subject is measured by first electrically stimulating multiple points about the circumference of the renal artery (or parts thereof as discussed below) using one or more electrodes as described above. As noted, the electrical stimulation at the multiple points about the circumference of the renal artery may be equally spaced about the circumference of the renal artery to lessen any chance of damage to the artery and/or the nerves due to over-stimulation. The renal artery may be stimulated about its circumference at 2, 3, 4, 5, 6, 7, 8 or more points to increase the likelihood that the efferent and afferent nerves are sufficiently electrically stimulated so as to provide the desired physiological response. In one particular embodiment, the electrical stimulation occurs at a single point within each of the four quadrants of the renal artery, such that there are four electrical stimulation points (corresponding to the four quadrants of the renal artery) about the circumference of the renal artery, that may be desirably equally spaced apart about the circumference.

The stimulation parameters to set include the current, pulse width, and frequency. At a particular fixed setting, the stimulation as described herein is generally performed for a time period of from about 60 seconds to about 120 seconds. The current and pulse rate can be varied to bring about the desired physiological response from the patient. The methods of the present disclosure provide improved reliability and consistency due to the fact that multiple points about the circumference of the renal artery are electrically stimulated (either sequentially or simultaneously) to elicit the desired physiological response. As discussed herein, by electrically stimulating multiple points about the circumference of the renal artery, there is a significantly increased confidence level that the desired afferent and/or efferent renal artery nerves are stimulated to elicit the desired physiological response.

There are numerous sites within the renal artery that are suitable for the electrical stimulation to occur. Suitable sites within the renal artery include, for example, unilateral sites, bilateral sites, the length of the renal artery, near the ostium, near the bifurcation, renal arterial branches distal to the bifurcation, intra-renal artery branches, and combinations thereof. Prior to stimulation, in some cases the doctor or operator may monitor the blood pressure of the patient over a period of time from about 60 seconds to 120 seconds so as to understand the blood pressure patterns and typical variation in blood pressure prior to electrical stimulation. In many cases, the more the baseline blood pressure rises as a result of the electrical stimulation, the more effective a renal denervation procedure may be for the patient as this in indicative of active nerve pathways in the renal artery.

In some embodiments, the electrical stimulation within the renal artery may be performed using sequential (multiplexing) electrical stimulation; that is, the electrical stimulation provided to the nerves may be sequential in nature wherein one electrode is energized, followed by the second, then the third, then the fourth, etc. Also, electrodes may be energized in pairs such that a first pair is energized followed by a second pair, etc. When sequential electrical stimulation is employed, the physiological response measured is the cumulative physiological response generated by the electrical stimulation in total as the sequential firing is done very quickly as described herein. In other embodiments, the electrical stimulation within the renal artery may be performed using simultaneous electrical stimulation wherein all electrical stimulation electrodes are energized at the same time such that the electrical stimulation to all four quadrants of the artery is provided as the same time. In some embodiments as described herein, sequential (multiplexing) electrical stimulation may be desirable as this type of electrical stimulation may result in a reduced chance of over-stimulation of the nerves.

The electrical stimulation provided to the renal artery by the electrodes generally provides a current in an amount of from about 0.1 milliamps to about 40 milliamps, including from about 10 milliamps to about 25 milliamps. This electrical current is supplied at a frequency of from about 1 Hertz to about 50 Hertz, including from about 15 Hertz to about 20 Hertz. The electrical current is generally supplied for a time period of from about 1 millisecond to about 25 milliseconds, including from about 1 millisecond to about 5 milliseconds, including from about 1 millisecond to about 2 milliseconds. The time between electrical stimulations may be from about 1 millisecond to about 10 milliseconds. In some embodiments, the electrical stimulation provided to the subject may be monopolar in nature (monopolar mode) between the electrical stimulation electrode and the indifferent electrode patch placed on the subject during a renal denervation procedure. In other embodiments, the electrical stimulation provided to the subject may be bipolar in nature (bipolar mode) between the electrical stimulation electrode and the adjacent ablation electrode or between two ablation electrodes. Because renal nerves generally traverse primarily longitudinally along the length of the renal artery, in many embodiments bipolar electrical stimulation may be desirable and effective in depolarizing the nerves.

The electrical stimulation described herein may be delivered to the subject as rectangular pulse waves, triangular pulse waves, sinusoidal pulse waves, Gaussian pulse waves, and combinations thereof. The electrical stimulation wave forms may include monophasic, charge balanced, and imbalanced biphasic, all of which may be delivered with our without delay. In some embodiments, it is desirable to provide the electrical stimulation in the form of rectangular pulse waves in a multiplexing manner from the electrode pairs.

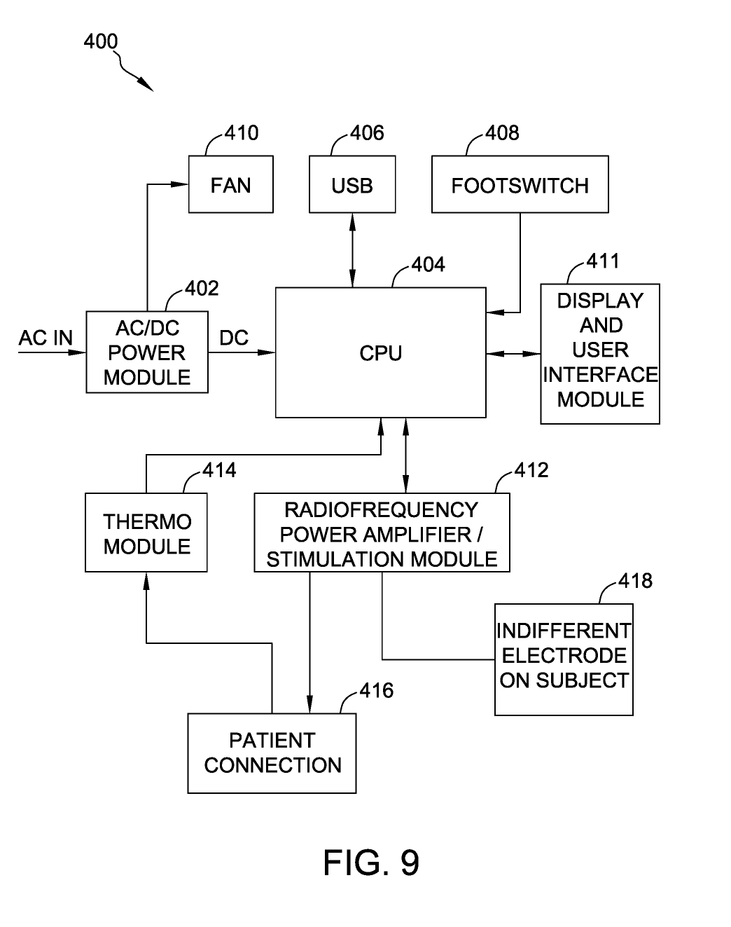

In some embodiments of the present disclosure, it may be desirable to integrate the electrical stimulation functionality into the radiofrequency or ultrasound generator, or any other generator with an energy source, that is utilized to provide the ablation aspects of the renal denervation procedure so that only a single generator unit is required for the procedure. Referring now to FIG. 9, there is shown one exemplary radiofrequency ablation generator block diagram with an electrical stimulation module integrated therein. FIG. 9 illustrates the circuits in one embodiment that would allow the electrical stimulation functionality into a radiofrequency system. Specifically system 400 may include AC/DC power module 402 for accepting AC power in that provides DC power to a central processing unit (CPU) 404 that is in electrical communication with a USB port 406, Footswitch 408 and a display and user interface module 411. AC/DC power module 402 is also in communication with fan 410. Central processing unit 404 is further in electrical communication with a radiofrequency power amplifier/stimulation module 412 for supplying radiofrequency energy and electrical energy to the electrodes of an ablation catheter (not shown in FIG. 9), and thermo module 414. A patient connection 416 is in electrical contact with thermo module 414 and radiofrequency power amplifier/stimulation module 412. An indifferent electrode patch on the subject 418 is also in electrical communication with radiofrequency power amplifier/stimulation module. Of course, the electrical stimulation function as described herein may be provided to the subject in any number of other ways and methods in accordance with the present disclosure.

Once the baseline physiological response of the subject has been measured through the electrical stimulation of the multiple points about the circumference of the renal artery as described above and a sufficient time has passed to allow for the physiological response to return to normal (generally at least 1 minute, or 2 minutes, or 3 minutes, or 4 minutes or even 5 minutes), the operator may then perform the renal denervation procedure on the renal artery of the subject. In some cases, the operator may determine that the electrical stimulation of the renal artery did not elicit a suitable physiological response from the subject and may determine that the particular subject is not a good candidate for a renal denervation procedure; that is, the methods of the present disclosure may also allow an operator to screen subjects prior to the renal denervation procedure to determine whether the particular subject may benefit from the renal denervation procedure.

After the operator has completed the renal denervation procedure in the renal artery, the renal artery is then again electrically stimulated at multiple points about its circumference as described herein and the physiological response of the subject determined. Generally, it is desirable to provide the electrical stimulation after renal denervation in the same area where the electrical stimulation was provided to determine the baseline physiological response. It should be noted, however, that the stimulation after renal denervation could be made at a different location. The operator can then compare the baseline physiological response (that is, the physiological response generated by the subject prior to any renal denervation due to the electrical stimulation) to the physiological response generated after the renal denervation procedure and determine the effectiveness of the renal denervation procedure. This intraprocedural feedback then allows the operator to determine, in real time and based on the physiological responses of the subject, whether any additional renal denervation may benefit the subject or whether the procedure should be ended. In the event that the operator determines that additional renal denervation may benefit the subject, a further renal denervation procedure may be done and another subsequent physiological response based on electrical stimulation performed and again evaluated against the baseline physiological response. In some embodiments, it may be beneficial for a second renal denervation procedure to be performed if the blood pressure of the subject in response to the electrical stimulation has not been reduced by at least about 20%, or even at least about 30%, or even at least about 40%, or even at least about 50%, without causing hypotension. In many embodiments, a second renal denervation procedure may be desirable if the blood pressure on the subject in response to the electrical stimulation has not been reduced by at least about 10% after the first renal denervation procedure.

### Example 1

In this example electrical stimulation (without any subsequent ablation) was applied to a canine at different points about the circumference of the renal artery to judge the effectiveness of the electrical stimulation at these different points to elicit a measured physiological response. An ablation catheter including an electrode basket similar to that of FIG. 2 and including four pairs of electrodes (2 electrodes on each of 4 splines) with each pair of electrodes including an ablation electrode and an electrically stimulating electrode was utilized in combination with an EnligHTN Generator (St. Jude Medical, St. Paul, Minn.) modified as shown in FIG. 9 to add the stimulation module. The Generator was used to first electrically stimulate points in all four quadrants of the most distal section of the bifurcation while remaining in the renal artery of a canine (age 1 year old) to determine the change in systolic, diastolic, and mean blood pressure after electrical stimulation as opposed to a normal blood pressure without electrical stimulation. The Generator was then used to electrically stimulate each quadrant individually to determine the change in systolic, diastolic, and mean blood pressure after electrical stimulation in the single quadrant as opposed to a normal blood pressure without electrical stimulation. The distance between the ablation electrode and the electrically stimulating electrode on each spline was about 1.5 millimeters.

A renal sheath was engaged into the ostium of the renal artery of the canine and the electrode basket catheter was inserted into the sheath and placed into the most distal section of the bifurcation, while remaining inside the main renal artery. The catheter basket was expanded and tissue contact was confirmed with the EnligHTN Generator. The systemic blood pressure of the canine prior to any electrical stimulation was recorded. The stimulation parameters were set at a current of 15 milliamps with a pulse width of 5 milliseconds and a frequency of 20 Hertz for a duration of 2 minutes. For the first electrical stimulation that included stimulation of all of the four quadrants, sequential bipolar electrode pair electrical stimulation using monophasic rectangular pulse waves was utilized and the systemic blood pressure was recorded. After a waiting period of five minutes to allow the blood pressure to return to normal (that is, a blood pressure in the absence of any external electrical stimulation) and with the catheter in the same location, the electrical stimulation electrode on the first spline was energized at the same settings and duration to provide electrical stimulation to only the first quadrant, and the blood pressure recorded. After a waiting period of five minutes to allow the blood pressure to return to normal, the electrical stimulation electrode on the second spline was energized at the same settings and duration to provide electrical stimulation to only the second quadrant, and the blood pressure recorded. This process was then repeated for the third and fourth quadrants such that at the end of the electrical stimulation, data had been gathered on the blood pressure increase as compared to normal for the electrical stimulation of all four quadrants simultaneously (though sequential electrical stimulation as noted above) and for each individual quadrant alone.

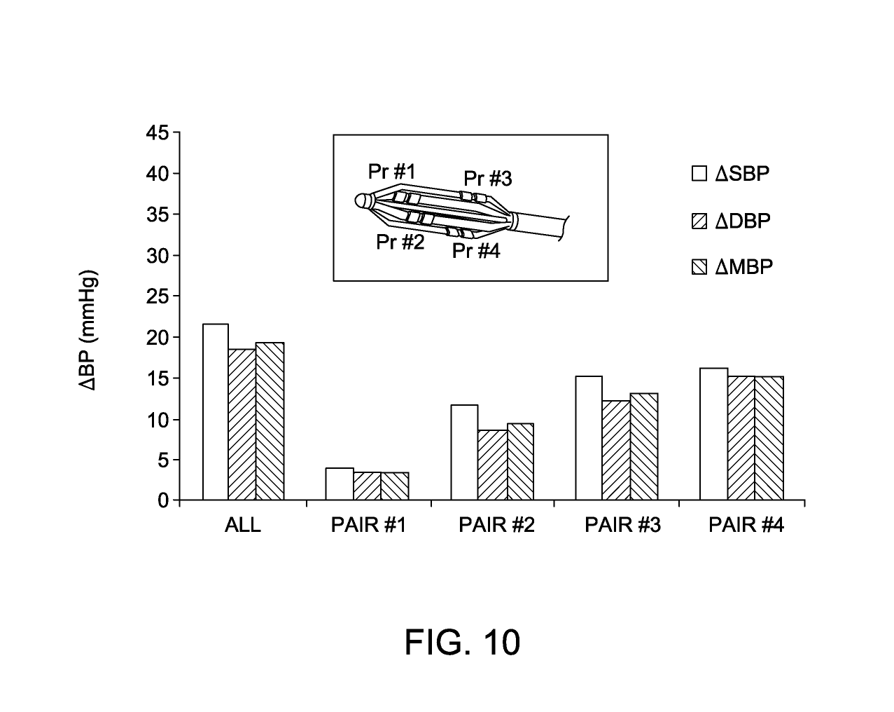

The results are shown in FIG. 10, which is a graph illustrating the change in blood pressure (for systolic, diastolic, and mean) for each of the five electrical stimulations (all four quadrants sequentially and each quadrant individually) as described above. As the FIG. 10 graph shows, when all four quadrants of the renal artery are electrically stimulated sequentially (ALL), there is a substantially greater increase in all three blood pressure measurements (SBP, DBP, and MBP) as compared to the electrical stimulation of any individual single quadrant in the renal artery (See FIG. 10 graph, PAIR #1, #2, #3, or #4). This indicates that sequential stimulation of all four quadrants of the renal artery (as compared to the electrical stimulation of any one quadrant alone) results in a high level of confidence that the desired nerves will be activated and a physiological response will be generated and amplified.

### Example 2

In this example an ablation catheter including an electrode basket similar to that of FIG. 2 and including four pairs of electrodes (2 electrodes on each of 4 splines) with each pair including an ablation electrode and an electrically stimulating electrode was utilized in combination with an EnligHTN Generator (St. Jude Medical, St. Paul, Minn.) modified as shown in FIG. 9 to add the stimulation module. The Generator was used to first electrically stimulate the four quadrants of the most distal section of the bifurcation while remaining in the renal artery of a canine (age 1 year old) to determine the change in systolic, diastolic, and mean blood pressure after stimulation (baseline blood pressure) as opposed to a normal blood pressure. The distance between the ablation electrode and the electrically stimulating electrode on each spline was 1.5 millimeters. After a sufficient time period to allow the blood pressure to return to normal (that is, a blood pressure in the absence of any external electrical stimulation), one or more renal denervation procedures were performed followed by electrical stimulation of the four quadrants of the renal artery after each renal denervation procedure to determine the effectiveness of the renal denervation procedure.

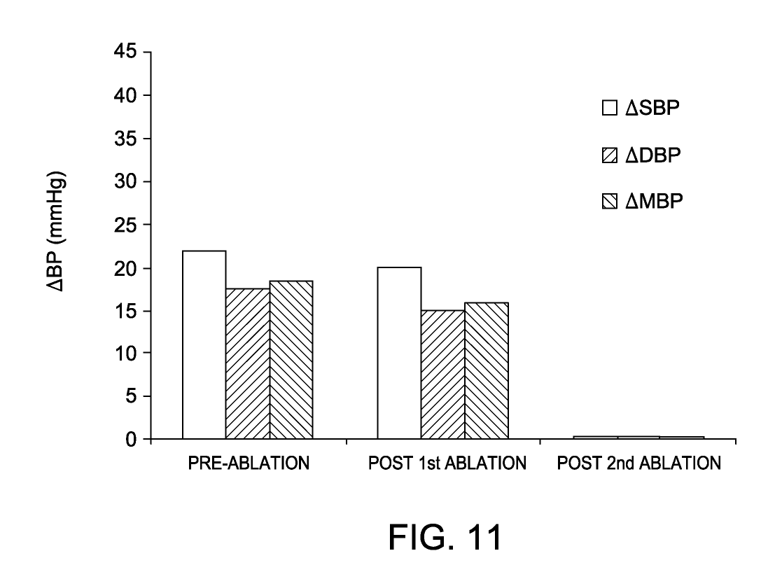

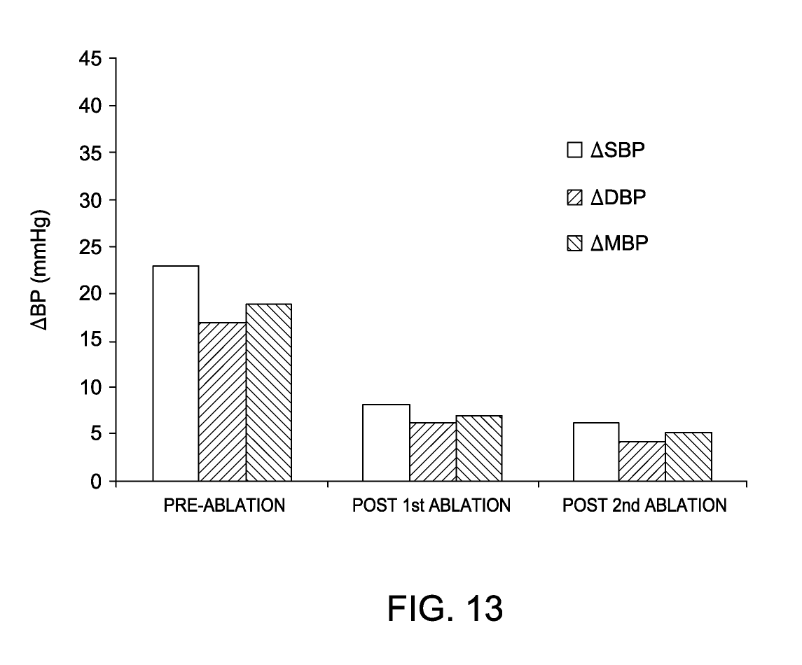

A renal sheath was engaged into the ostium of the renal artery (Left Renal Artery for Cases #1 and #2 and Right Renal Artery for Case #3) of the canine and the electrode basket catheter was inserted into the sheath and placed into the most distal section of the bifurcation, while remaining inside the main renal artery. The catheter basket was expanded and tissue contact was confirmed with the EnligHTN Generator. The normal systemic blood pressure prior to any electrical stimulation was recorded. Afterwards, with the stimulation parameters set at a current of 15 milliamps, a pulse width of 5 milliseconds, and a frequency of 20 Hertz, sequential bipolar electrode pair electrical stimulation using monophasic rectangular pulse waves was utilized and the systemic blood pressure recorded. After a waiting period of at least five minutes to allow the blood pressure to return to normal, a renal denervation procedure was conducted on the canine. The catheter was moved proximally towards the aorta and the renal denervation procedure was conducted with the EnligHTN Generator at settings of 8 watts, 70 degrees Celsius, and 60 seconds. After the renal denervation procedure was complete, and after a waiting period of five minutes to allow the blood pressure to return to normal, the four electrical stimulation electrodes were again energized at the original stimulation location at the most distal main renal artery section near the bifurcation as described above and the blood pressure recorded to determine whether the renal denervation procedure was successful. In some cases (Case #1 (FIG. 11) and Case #3 (FIG. 13)) a second renal denervation procedure was then carried out at a separate second location within the main renal artery followed again by electrical stimulation at the most distal main renal artery section near the bifurcation to determine the effectiveness of the renal denervation procedure.

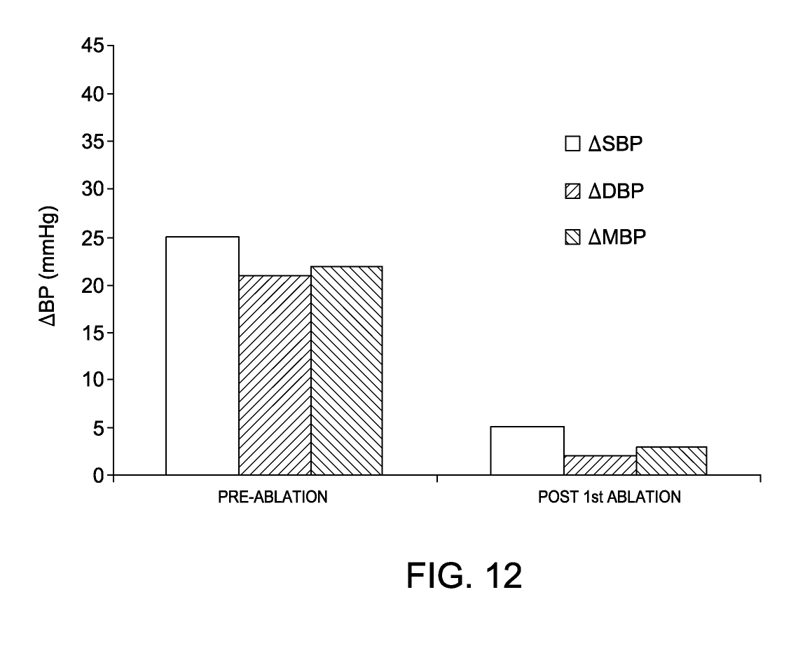

The results are shown in FIGS. 11, 12, and 13, which are graphs illustrating the change in blood pressure (for systolic, diastolic, and mean) before and after one or two renal denervation procedures. As the graphs show, one or two renal denervation procedures substantially decrease the resulting blood pressure increase of the canine. And specifically, the graphs show that the electrical stimulation utilized on the canine is sufficient to indicate the procedural progress of the renal denervation, and whether additional denervation may be beneficial.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method of evaluating the intraprocedural success of a renal denervation procedure on a subject, the method comprising:
electrically stimulating multiple points about the circumference of a renal artery of the subject to determine a baseline physiological response of the subject;
performing a renal denervation procedure on the renal artery of the subject;
electrically stimulating the multiple points about the circumference of the renal artery of the subject to determine the physiological response of the subject;
comparing the baseline physiological response of the subject prior to renal denervation with the physiological response after renal denervation.

2. The method of claim 1 further comprising determining whether to perform additional renal denervation on the subject by using the compared physiological responses.

3. The method of claim 1 wherein the electrical stimulation of the renal artery and the renal denervation are performed using the same ablation catheter.

4. The method of claim 3 wherein the ablation catheter comprises at least four ablation electrodes and at least four electrical stimulation electrodes.

5. The method of claim 3 wherein the ablation catheter is a balloon catheter.

6. The method of claim 3 wherein the ablation catheter comprises at least four ablation electrodes.

7. The method of claim 4 wherein the distance between each ablation electrode and each electrical stimulation electrode is from about 0.5 millimeters to about 2.5 millimeters.

8. The method of claim 4 wherein the ablation electrodes and the electrical stimulation electrodes are positioned and configured on the at least four splines.

9. The method of claim 4 wherein the electrical stimulation is performed using sequential electrical stimulation.

10. The method of claim 4 wherein the electrical stimulation is performed using simultaneous electrical stimulation.

11. The method of claim 1 wherein the electrical stimulation is a monopolar electrical stimulation.

12. The method of claim 1 wherein the electrical stimulation is bipolar electrical stimulation.

13. The method of claim 1 wherein the physiological response of the subject measured is selected from the group consisting of blood pressure, heart rate, renal blood flow rate, catecholamine level, heart rate variability, natriuresis, other biomarker levels, and combinations thereof.

14. The method of claim 13 wherein the physiological response of the subject measure is blood pressure.

15. The method of claim 1 wherein the electrical stimulation is in the form of rectangular pulse waves.

16. The method of claim 1 wherein the electrical stimulation provides current in an amount of from about 0.1 milliamps to about 40 milliamps at a frequency of from about 1 Hertz to about 50 Hertz for a time period of from about 1 millisecond to about 5 milliseconds.

17. The method of claim 16 wherein the electrical stimulation provides current in an amount of from about 10 milliamps to about 25 milliamps at a frequency of from about 15 Hertz to about 20 Hertz for a time period of from about 1 millisecond to about 5 milliseconds.

18. A method of determining the end point of a renal denervation procedure on a subject, the method comprising:
electrically stimulating the four quadrants of a renal artery of the subject through electrical stimulation to determine a baseline reading of the subject's blood pressure using an ablation catheter comprising at least four ablation electrodes and at least four electrical stimulation electrodes and configured to electrically contact the four quadrants of the renal artery, wherein the distance between each ablation electrode and each electrical stimulation electrode in the pairs is from about 0.5 millimeters to about 2.5 millimeters;
performing a renal denervation procedure on the renal artery of the subject using the ablation catheter;
electrically stimulating the four quadrants of the renal artery of the subject through electrical stimulation using the ablation catheter configured to electrically contact the four quadrants of the renal artery and determine the subject's blood pressure;
comparing the baseline blood pressure of the subject prior to renal denervation with the blood pressure after renal denervation; and
determining whether to perform an additional renal denervation procedure.

19. The method of claim 18 wherein the electrical stimulation provides current in an amount of from about 10 milliamps to about 25 milliamps at a frequency of from about 15 Hertz to about 20 Hertz for a time period of from about 1 millisecond to about 5 milliseconds.

20. The method of claim 18 wherein the electrical stimulation is bipolar electrical stimulation.
